(19) United States
(12) Patent Application Publication
(10) Pub. No.: US 2026/0298865 A1
(43) Pub. Date: Oct. 1, 2026

(54) ALUMINUM ION SENSOR AND METHOD TO DETECT ALUMINUM ION CONCENTRATION

(71) Applicant: THE AMERICAN UNIVERSITY IN CAIRO, New Cairo (EG)

(72) Inventors: Nada ALI, New Cairo (EG); Tamer SHOEIB, New Cairo (EG); Norhan BADRELDIN, New Cario (EG)

(73) Assignee: THE AMERICAN UNIVERSITY IN CAIRO, New Cairo (EG)

(21) Appl. No.: 19/552,676

(22) Filed: Feb. 27, 2026

Related U.S. Application Data
(60) Provisional application No. 63/764,007, filed on Feb. 27, 2025.

(51) Int. Cl. G01N 27/333; G01N 27/30; G01N 27/413; G01N 33/14; G01N 33/49

(52) U.S. Cl. CPC G01N 27/3335; G01N 27/301; G01N 27/413; G01N 33/14; G01N 33/49

(57) ABSTRACT

A screen-printed sensor for detecting aluminum ions in food and biological samples is provided. The sensor includes an aluminum ion-selective membrane containing quercetin as an aluminum ionophore and multi-walled carbon nanotubes. The sensor provides a low-cost point of analysis method to determine aluminum ion concentration in a range of from 0.008 to 1070 μM in food and beverage materials, reflecting the general detection capability of the sensor under aqueous conditions.

### TECHNICAL FIELD

The present disclosure relates to a low-cost portable sensor for detecting aluminum ions, which has a high selectivity for Al³⁺, a rapid response time, pH stability in a selected pH range, and detection limits commensurate with requirements for analysis of food, beverage, and biological samples.

### BACKGROUND

The potential health risks associated with aluminum intake through food, beverages, and other environmental sources have created a need for an accurate Al³⁺ detection method, which can be employed in various fields, including food safety monitoring and medical diagnostics. The need is directed to suitability for point-of-care settings, affordability, ease of use, and rapid, accurate analysis capabilities. Conventional methods for detecting aluminum in real samples, such as flow injection analysis (FIA), graphite furnace atomic absorption spectrometry (GFAAS), and inductively coupled plasma atomic emission spectrometry (ICP-AES), while highly accurate, require specialized equipment, skilled operators, and significant financial investments. These factors limit their accessibility and practicality, especially in resource-constrained settings.

The successful example of electrochemical sensors for the point-of-care monitoring of glucose based on modified one-use disposable electrodes characterized by low cost, ability to mass produce, reproducibility, and simplicity has set the stage for similar sensor development of other types of disposable strips produced via screen-printing technology. Screen-printed electrodes have been employed in wide and diverse applications, for example, cholesterol biosensors, determination of 4-hexylresorcinol in pharmaceutical products, the one-step enzyme immobilization of glucose oxidase with silica sol-gel/polyvinyl alcohol hybrid films, food allergen detection, determination of serum alcohol, and cysteine sensing at poly(3,4-ethylenedioxythiophene)-modified screen-printed electrodes.

Thus, there is a need for an Al³⁺ detection system that can be employed in screen-printed sensors, which are useful for point-of-care settings, can be produced in large quantities at an affordable cost, do not require high technical expertise to operate, and provide rapid, accurate analysis capabilities.

### SUMMARY OF THE DISCLOSURE

These and other objects are provided by the present disclosure, the first embodiment of which includes a solid-contact Al ion selective electrode, comprising: a substrate; a layer of an electrically conductive carbon material applied to a surface of the substrate; and an ion selective membrane in contact with the layer of electrically conductive carbon material, the ion selective membrane comprising: quercetin; multi-walled carbon nanotubes (MWCNT's); a plasticizer; a polymer; and an ion exchange material, wherein the MWCNT's form a network of interconnected fibers at a surface of the membrane.

In one aspect of the first embodiment, the MWCNT's network of interconnected fibers is an electrical double layer.

In one aspect of the first embodiment, the ion exchange material is lipophilic, having a lipophilicity value log P of 10 or greater, and in one particular aspect, the ion exchange material is potassium tetrakis(4-chlorophenyl)borate (KTCPB).

In one aspect of the first embodiment, the plasticizer has a dielectric constant of 10 or less, and in one particular aspect, the plasticizer is nitrophenyl octyl ether (NOPE).

In one aspect of the first embodiment, the substrate is selected from the group consisting of glass, a polymer sheet, and a metal foil.

In one aspect of the first embodiment, the electrically conductive carbon material comprises one or more selected from the group consisting of carbon black, carbon nanotubes, graphite, and graphene, and in a further aspect, the layer of an electrically conductive carbon material comprises from 60 wt % to 80 wt % of the electrically conductive carbon material.

In one aspect of the first embodiment, the polymer contained in the ion-selective membrane is selected from the group consisting of polyethylene, polypropylene, and polyvinyl chloride.

In one aspect of the first embodiment, the ion selective membrane comprises: from 0.25 wt % to 5.0 wt % quercetin; from 2.0 wt % to 10 wt % of the multi-walled carbon nanotubes (MWCNT's); from 30 wt % to 60 wt % of the plasticizer; from 20 wt % to 50 wt % of the polymer; and from 0.05 wt % to 0.50 wt % of the ion exchange material.

In a second embodiment, the present disclosure provides a potentiometric aluminum ion sensor, comprising: a substrate; a screen printed solid-contact Al ion selective electrode on the substrate; a screen printed Ag/AgCl reference electrode electrically isolated from the solid-contact Al ion selective electrode on the substrate; and a potentiometer, wherein each electrode is in electrical contact with the potentiometer, the screen printed solid-contact Al ion selective electrode comprises: a layer of an electrically conductive carbon material applied to a surface of the substrate; an ion selective membrane in contact with the layer of electrically conductive carbon material, the ion selective membrane comprising: quercetin; multi-walled carbon nanotubes (MWCNT's); a plasticizer; a polymer; and an ion exchange material, wherein the MWCNT's form a network of interconnected fibers at a surface of the membrane which is a capacitive/electrical double-layer (EDL) transducer that stabilizes an electrode potential, and the solid-contact Al ion selective electrode is free of an internal filling solution.

In an aspect of the second embodiment, the ion exchange material is lipophilic, having a lipophilicity value log P value of 10 or greater, and in one particular aspect, the ion exchange material is potassium tetrakis(4-chlorophenyl)borate (KTCPB).

In an aspect of the second embodiment, the plasticizer has a dielectric constant of 10 or less, and in one particular aspect, the plasticizer is nitrophenyl octyl ether (NOPE).

In an aspect of the second embodiment, the electrically conductive carbon material comprises one or more selected from the group consisting of carbon black, carbon nanotubes, graphite and graphene, and in a further aspect, the layer of an electrically conductive carbon material comprises from 60 wt % to 80 wt % of the electrically conductive carbon material.

In one aspect of the second embodiment, the polymer contained in the ion-selective membrane is selected from the group consisting of polyethylene, polypropylene, and polyvinyl chloride.

In one aspect of the second embodiment, the ion selective membrane comprises: from 1.0 wt % to 5.0 wt % quercetin; from 2.0 wt % to 10 wt % of the multi-walled carbon nanotubes (MWCNT's); from 30 wt % to 60 wt % of the plasticizer; from 20 wt % to 50 wt % of the polymer; and from 0.05 wt % to 0.50 wt % of the ion exchange material.

In a third embodiment, the present disclosure provides a method for detecting aluminum ion concentration in a sample to be analyzed, comprising: digesting the sample in a mixture of nitric acid and hydrogen peroxide followed by microwave digestion; contacting the digested sample with the potentiometric aluminum ion sensor according to the second embodiment to obtain a potential emf value; comparing the obtained emf value to a calibration curve of emf value versus log aluminum ion (Al³⁺) in M to determine the Al³⁺ M value of the sample.

In an aspect of the third embodiment, the sample to be analyzed is a food or a beverage.

In an aspect of the third embodiment, a concentration range of Al³⁺ ion is from 0.008 to 1070 μM.

In an aspect of the third embodiment, the response time of the potentiometric aluminum ion sensor to obtain the Al³⁺ concentration is from 3 to 10 seconds.

In a fourth aspect, the present disclosure provides a method for detecting aluminum ion concentration in a biological fluid to be analyzed, comprising: adjusting the pH of the biological fluid to 4.5; contacting the pH adjusted sample with the potentiometric aluminum ion sensor according to the second embodiment to obtain a potential emf value; comparing the obtained emf value to a calibration curve of emf value versus log aluminum ion (Al³⁺) in M to determine the Al³⁺ M value of the sample.

In an aspect of the fourth embodiment, the concentration range of Al³⁺ ions is from 0.023-100 μM in biological fluids

In an aspect of the fourth embodiment, the response time of the potentiometric aluminum ion sensor to obtain the Al³⁺ concentration according to the matrix is from 6 to 10 seconds.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A more complete appreciation of the disclosure and many of the attendant advantages thereof will be readily obtained as the same becomes better understood by reference to the following detailed description when considered in connection with the accompanying drawings, wherein:

FIG. 1 shows the method to prepare an ion-selective membrane employed in Example 2.

FIG. 2 shows the method to construct the liquid-contact sensor employed in Example 2.

FIG. 3 is a bar chart showing performance characteristics of the liquid contact sensors of Example 2.

FIG. 4 shows a schematic drawing of the fabrication process described in Example 3.

FIG. 5 shows a comparative schematic picture of the method to prepare the unmodified screen-printed electrode (SPE) and the modified SPE described in Example 4.

FIG. 6 shows a profile of the measured potential in mV versus log concentrations of aluminum in M obtained with MWCNT/ISM and MWCNT-free sensor as described in example 4.

FIG. 7 shows the reversibility of the MWCNT modified sensor observed as Al³⁺ concentrations alternate between indicated values.

FIG. 8 shows a comparison of the long-term stability of the MWCNT-modified sensor and the unmodified sensor obtained in example 4.

FIG. 9 shows a water layer test of the MWCNT-modified sensor and the MWCNT-free sensor obtained in example 4.

FIG. 10 shows the short-term potential stability of the MWCNT microfabricated sensor and the MWCNT free one obtained in example 4 upon measuring in a 0.10 mM aluminum solution for one hr.

FIG. 11 shows a scale diagram of a potentiometric aluminum ion sensor according to one embodiment of the present disclosure.

FIG. 12 shows a 1H NMR spectrum of a quercetin sample according to an embodiment of the disclosure.

FIG. 13 shows an analysis focused on the spectrum of FIG. 12

FIG. 14 shows an expansion of the region from 2.5 ppm to 6.6 ppm of the spectrum of FIG. 12.

FIG. 15 shows an expansion of the region from 6.1 ppm to 7.8 ppm of the spectrum of FIG. 12.

FIG. 16 shows an expansion of the region from 9.0 ppm to 13 ppm of the spectrum of FIG. 12.

FIG. 17 shows an FTIR spectrum of a quercetin sample according to an embodiment of the disclosure.

FIG. 18 shows an XRD analysis of a quercetin sample according to an embodiment of the disclosure.

FIG. 19 shows the calibration of spiked simulated plasma standards spanning 1.0×10⁻⁸ to 1.0×10⁻³ M Al³⁺ at pH 4.5±0.1 described in Example 8.

FIG. 20 shows a profile of the measured potential in mV versus log concentrations of aluminum in M obtained with MWCNT/ISM described in Example 8.

FIG. 21 shows the reversibility of MWCNT modified sensor observed as Al³⁺ concentrations alternate between the indicated values described in Example 8.

FIG. 22 shows the long-term stability and reproducibility of the sensors described in Example 8.

FIG. 23 shows the effect of pH on the potential stability of the MWCNT-sensor in aluminum solution as described in Example 8.

### DETAILED DESCRIPTION OF THE PREFERRED EMBODIMENTS

In the following description, the words “a” and “an” and the like carry the meaning of “one or more.” The phrases “selected from the group consisting of,” “chosen from,” and the like include mixtures of the specified materials. Terms such as “contain(s)” and the like are open terms meaning ‘including at least’ unless otherwise specifically noted. All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

To address the above-listed objects, the present inventors focused research on developing a novel, cost-effective, and environmentally friendly approach to produce Al³⁺ sensors based on ion-selective electrodes, which offer advantages including rapid response time, high selectivity, and sensitivity to Al³⁺, suitability for point-of-care applications, and applicability to food and beverage quality control and Al³⁺ analysis of biological samples.

One major challenge in developing aluminum ion-selective electrodes lies in the selection of a suitable Al³⁺ ionophore. Traditionally, synthetic ionophores have been used, and they also often suffer from limitations such as low selectivity and stability. To overcome these drawbacks, the inventors have surprisingly discovered that the compound quercetin (Formula 1) has a unique molecular structure having multiple electron-rich sites which may form stable complexes with aluminum ions, resulting in enhanced selectivity and sensitivity as an Al³⁺ sensor ionophore.

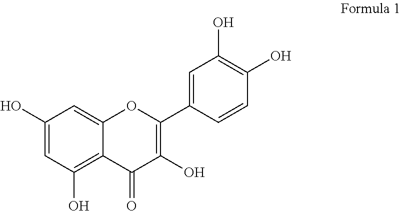 [SMILES: O=c1c(O)c(-c2ccc(O)c(O)c2)oc2cc(O)cc(O)c12]

Quercetin is a natural dietary flavonoid found in fruits, flowers, and vegetables, which is employed as an antioxidant supplement. However, utility as an ionophore having high selectivity and sensitivity for Al³⁺ ions is not generally known.

The use of quercetin, which is a biodegradable natural product, enhances the eco-friendly character of the present disclosure. Quercetin is a bio-sustainable resource that can be extracted from the leaves and stems of many plants, including elderberry leaves, as described in the Examples.

The performance of Quercetin as an Al³⁺ selective ionophore in comparison to other possible ionophores was demonstrated by preparing liquid contact electrodes as described in Example 2 with the samples prepared, shown in Table 1. In preparing the membranes, potassium tetrakis(4-chlorophenyl)borate (KTCPB) was chosen as the ion exchanger due to its high lipophilicity (log P=10.4), which improved the electrode's selectivity. Nitrophenyl octyl ether (NOPE) was selected as the plasticizer due to its high dielectric constant, which aids in charge retention and improves the electrode's response. Methyl tetrahydrofuran (MeTHF) was used as a solvent in the membrane fabrication process, offering environmental benefits over traditional solvents like tetrahydrofuran (THF).

MeTHF is synthesized in adherence to the seventh principle of Green Chemistry, whereby it is derived from renewable agricultural by-products. MeTHF was reportedly synthesized from renewable agricultural by-products with a lower life cycle footprint, through catalytic reduction of levulinic acid and furfural that are produced by the dehydration of five-carbon sugars present in corncob biomass. Various evaluation studies have demonstrated that the production of 1 kg of MeTHF results in greenhouse gas emissions of 0.150 kg of CO₂, whereas the manufacturing of THF emits 5.46 kg of CO₂. This discrepancy indicates that MeTHF offers a more environmentally friendly alternative, leading to fewer environmental consequences and potential benefits for human health.

The fabricated liquid-contact sensors were evaluated based on linearity range, slope, response time, and selectivity. As shown in Table 2 of Example 2, the quercetin-based ion-selective electrode (ISE) exhibited superior performance in terms of signal stability, sensitivity, and linear range compared to the other ionophores evaluated. The findings of Table 2 indicate that electrodes with the lowest detection limits can be achieved by utilizing highly lipophilic ion exchangers, incorporating quercetin ionophores, and NOPE as a plasticizer. The optimized quercetin-based ion-selective membrane (ISM) was selected as the membrane composition for further development.

In addition, the strong binding affinity between quercetin and aluminum ions contributed to the enhanced performance by minimizing aluminum ion release from the membrane into the sample.

The inventors recognized that ion-selective electrodes (ISEs) utilizing liquid contact sensors have significant limitations regarding miniaturization, stability, and maintenance. Therefore, to proceed to develop a portable and environmentally friendly potentiometric sensor specifically designed for the detection of aluminum ions in food and beverages, to address these shortcomings, solid-contact screen-printed electrodes were investigated. Screen-printed electrodes are a simple and cost-effective method for fabricating electrochemical sensors. However, it is known that solid-contact electrodes are prone to water accumulation, which can result in potential instabilities and a reduced sensor lifespan.

To address and mitigate this water accumulation issue, multi-walled carbon nanotubes (MWCNTs) were introduced into the ion-selective membrane as a transducer layer within the screen-printed electrodes. MWCNTs have unique electrical and thermal properties and can be deposited on various surfaces. The MWCNTs are chemically inert, have a wide pH range of stability, and are hydrophobic and thus effective in enhancing the performance of the Al³⁺ ion-selective electrode. The MWCNTs form a network of interconnected fibers on the electrode surface, which serves as an ion-to-electron transduction layer, creating an electrical double layer (EDL) transducer that stabilizes the potential and ensures reliable signal transmission. This layer also prevents water layer formation at the ion-selective membrane surface. A performance comparison of ion sensors with and without MWCNTs is described in Example 4. General fabrication of screen-printed electrodes is described in Example 3.

As shown in Table 3 in Example 4, both the modified and unmodified Screen-Printed Solid Contact Electrodes (SPEs) showed good reversibility, with minimal hysteresis effects. However, the MWCNT-modified SPE exhibited superior performance compared to the unmodified SPE. The modified SPE demonstrated a broader linear range (0.023 μM to 1070 μM) compared to the unmodified SPE (1.07 μM to 1070 μM). The modified SPE showed a steeper Nernstian slope, indicating higher sensitivity and the modified SPE achieved a lower detection limit of 0.008 μM. Further, the modified SPE exhibited a faster response time, especially at lower concentrations.

Thus, an optimized Al ion sensing membrane was developed by incorporating quercetin as an ionophore onto the MWCNT-modified screen-printed electrodes. This strategic combination of quercetin and MWCNTs provides a synergistic performance effect to provide precise detection of Al³⁺, resulting in a sensor exhibiting exceptional stability, reproducibility, and sensitivity for the detection of aluminum ions across various food and beverage samples. By combining the advantages of ion-selective electrodes with the superior properties of quercetin, the present disclosure provides a solution for accurate and rapid aluminum detection in food and beverage samples.

The research was extended to explore the potential of the developed Al³⁺ sensor electrode for diagnostic applications. By evaluating the electrode on simulated blood plasma adjusted to pH 4.5, Al³⁺ ions were successfully detected within a concentration range of 1.0 to 10.0 μM. The recoveries for these concentrations were 94.51% and 90.9%, respectively, indicating the accuracy and reliability of the method. These results indicate that the sensor could be integrated into point-of-care diagnostic devices to detect aluminum levels in biological fluids, aiding in the diagnosis and treatment of aluminum toxicity.

Thus, in a first embodiment, the present disclosure provides a solid-contact Al ion-selective electrode. The electrode is constructed on a base substrate by applying a layer of an electrically conductive carbon material to the surface of the substrate. An ion-selective membrane containing a mixture of quercetin, multi-walled carbon nanotubes (MWCNT's), a plasticizer, a polymer, and an ion exchange material is deposited in contact with the layer of electrically conductive carbon material. As previously described, the MWCNT's form a network of interconnected fibers on the surface of the membrane and function as a transducer or electrical double layer.

As indicated by the description “solid contact,” the disclosed Al ion-selective electrode does not contain a filling solution.

The quercetin may be obtained by extraction from a plant source, for example, elderberry, as described in Example 1, or may be obtained from commercially available sources. Quercetin is generally obtained as a yellow crystalline powder having a melting point of 316° C.

Although various grades of MWCNT's are available, at a minimum quality, material having a carbon content of 90% carbon is employed, preferably 96% carbon, and most preferably 99% carbon. The length of the MWCNT's is from 2.5 to 20 μm, alternatively from 5 to 20 μm, and more preferably 10 to 20 μm. MWCNT's are commercially available and may be obtained from any source meeting the purity requirement.

The ion exchange material may be any known Al³⁺ ion exchange component which is lipophilic, having a lipophilicity value log P of 7 or greater, preferably 8 or greater, more preferably 9 or greater, and most preferably 10 or greater. Having a log P value of 10 or greater ensures optimal lipophilicity, which results in long-term membrane retention. Exemplary Al³⁺ ion exchange materials include sodium tetrakis-[3,5-bis(1,1,1,3,3,3-hexafluoro-2-methoxy-2-propyl)phenyl] borate (NaHFPB), sodium tetraphenylborate (NaTPB), tetrakis(4-fluorophenyl)borate (cesibor), tetrakis[3,5-bis(trifluoromethyl)phenyl]borate (TFPB), and potassium tetrakis(4-chlorophenyl)borate (KTCPB).

In the present disclosure, potassium tetrakis(4-chlorophenyl)borate (KTCPB) has been shown to be highly effective for membrane function and long term stability. However, this disclosure is not limited to KTCPB, and any Al³⁺ ion exchange component having high lipophilicity is included in the present disclosure.

The plasticizer may be any conventionally known plasticizer having a dielectric constant of 10 or less. In the present disclosure, nitrophenyl octyl ether (NOPE) has been shown to be effective. NOPE's moderate polarity supports ion-pairing mechanisms critical for the formation of charged ionophores and helps dissolve charged ionophores (e.g., quercetin) and ion-exchange materials (e.g., KTCPB) while maintaining membrane homogeneity.

A higher dielectric constant reduces the electrostatic forces between ions, facilitating their movement within the membrane.

NOPE is widely used in ISE designs due to its stability, low volatility, and compatibility with PVC membranes. However, this disclosure is not limited to NOPE. Other exemplary plasticizers that may be employed include, for example, Bis(2-ethylhexyl) sebacate (DOS, ε=~4) and Dibutyl phthalate (DBP, ε=~6.4)

The electrically conductive carbon material may be one or more selected from the group consisting of carbon black, carbon nanotubes, graphite, and graphene. The layer of electrically conductive carbon material may comprise a polymer binder. Generally, any conventionally known binder or combination of binders may be employed. For example, the polymer binder may be one or more selected from a polytetrafluoroethylene (PTFE), a polyvinyl alcohol (PVA), a polyvinylidene fluoride (PVDF), poly(3,4-ethylenedioxythiophene):poly[(4-styrenesulfonyl) (trifluoromethylsulfonyl)imide] (PEDOT:PSSTFSI), a sodium alginate (SA), a polyacrylic acid (PAA), a sodium carboxymethyl cellulose (CMC) and a styrene-butadiene rubber (SBR). However, the disclosure is not limited to these exemplary polymer binders.

The content of the electrically conductive carbon material in the layer of the electrically conductive carbon material may be from 60 wt % to 80 wt %, preferably from 60 wt % to 70 wt % for applications requiring enhanced mechanical stability, preferably from 65 wt % to 75 wt % for balanced conductivity and structural integrity, and preferably from 70 wt % to 80 wt % for high-conductivity applications, wherein the binder is selected to complement the carbon content.

The polymer binder contained in the ion-selective membrane may be one or more selected from polyethylene, polypropylene, and polyvinyl chloride. However, the disclosure is not limited to these polymers, and other conventionally known polymer binders may be employed.

Although in consideration of cost, ease of handling, and availability, the substrate is preferably a plastic, the substrate may be glass or a metal foil, as well as a polymer sheet. The substrate may be selected in consideration of the intended end-use requirements.

The composition of the ion-selective membrane may be varied to achieve desirable performance characteristics of the electrode depending on intended utility. The following disclosed ranges were empirically determined through iterative testing to maximize sensor performance (sensitivity, selectivity, and response time) while ensuring mechanical stability and reproducibility.

Generally, the composition of the ion-selective membrane includes from 0.25 wt % to 5.0 wt % quercetin, preferably from 0.5 wt % to 3.0 wt %, and most preferably from 1.0 wt % to 2.0 wt %. Having a content of 0.25 wt % or greater of quercetin ensures sufficient ionophore activity for Al³⁺ chelation. Below this threshold, sensitivity may decline significantly. A content of quercetin greater than 5.0 wt % may lead to membrane brittleness and phase separation as well as potential leaching and reduced sensor stability.

The content of the multi-walled carbon nanotubes (MWCNT's) may be from 2.0 wt % to 10 wt %, preferably 3.0 wt % to 9.0 wt %, and most preferably from 4.0 wt % to 8.0 wt %. When the content is at least 2.0 wt %, electrical conductivity for signal transduction is adequate. Maintaining an upper limit of 10 wt % avoids potential aggregation of the MWCNT's and excessive rigidity of the membrane.

The content of the plasticizer may be from 30 wt % to 60 wt %, preferably from 35 wt % to 58 wt % and most preferably from 40 wt % to 55 wt %. Maintaining the plasticizer content at 30 wt % or higher ensures membrane plasticity and ion mobility. Lower amounts than 30 wt % lead to rigid membranes having poor Al³⁺ sensitivity. If the plasticizer content exceeds 60 wt % the mechanical integrity of the membrane may deteriorate.

The content of the polymer binder may be from 20 wt % to 50 wt %, preferably 22.5 wt % to 45 wt %, and most preferably 25 wt % to 40 wt %. A polymer binder content of 20 wt % or higher provides a stable membrane structure. A content of less than 20 wt % may lead to membrane disintegration. Polymer binder content greater than 50 wt % may inhibit ion diffusion within the membrane.

The content of the ion exchange material may be from 0.05 wt % to 0.50 wt %, preferably from 0.08 wt % to 0.40 wt %, and most preferably from 0.1 wt % to 0.30 wt %. The content of ion exchange material in this range provides a balance of ion-exchange capacity and avoids membrane fouling.

Variation within these ranges may be applied based upon achieving further sensitivity, response time, electrode stability, and other performance parameters. In addition, compositions outside these ranges may be within the present disclosure.

In a second embodiment, the present disclosure provides a potentiometric aluminum ion sensor, constructed of a planar substrate, a screen-printed solid-contact Al ion selective electrode on the substrate, a screen-printed Ag/AgCl reference electrode electrically isolated from the solid-contact Al ion selective electrode on the substrate, wherein the electrodes are configured to be electrically connected to a potentiometer. The potentiometer may be portable, such that the measurements may be taken on site in a testing field outside a laboratory.

The screen-printed solid-contact Al ion-selective electrode has a layer of an electrically conductive carbon material applied to the surface of the substrate and an ion-selective membrane in contact with the layer of electrically conductive carbon material. The ion-selective membrane contains quercetin, multi-walled carbon nanotubes (MWCNT's), a plasticizer, a polymer, and an ion exchange material. The structure and composition of the screen-printed solid-contact Al ion selective electrode is the same as disclosed for the solid-contact Al ion selective electrode of the first embodiment, and that description is incorporated here by reference.

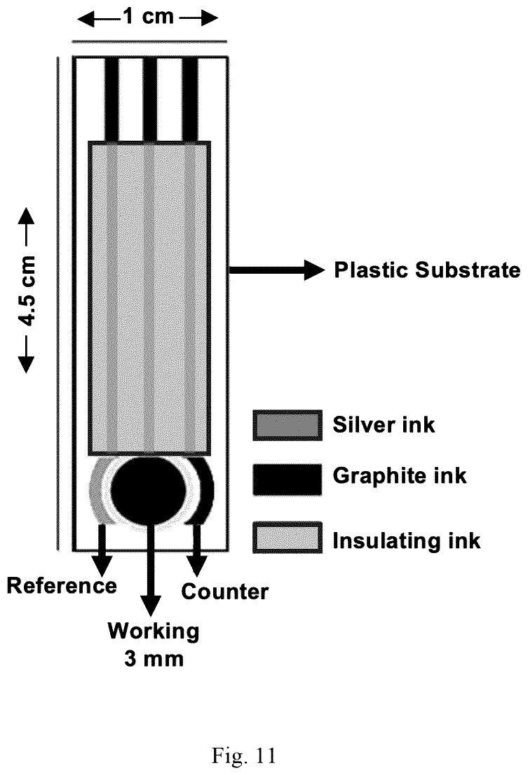

FIG. 11 shows a scale drawing of a potentiometric aluminum ion sensor according to an embodiment of the present disclosure.

The potentiometer may be any commercially available unit configured to contact and read the output of the aluminum ion sensor.

In a third embodiment, the present disclosure provides a method for detecting an aluminum ion concentration in a sample to be analyzed. According to the method, the sample to be analyzed is digested in a mixture of nitric acid and hydrogen peroxide, followed by microwave digestion. The pH of the digested sample is adjusted to a value of from 3.0 to 5.0 using a conventional acid or base. A portion of the digested sample is directly contacted with the potentiometric aluminum ion sensor previously described as the second embodiment, and a potential emf value is read from the potentiometer. The obtained emf value is compared to a calibration curve of emf value versus log aluminum ion (Al³⁺) in M to determine the Al³⁺ M value of the sample.

No purification of the digested and pH-adjusted sample is required prior to contacting the potentiometric aluminum ion sensor to conduct the measurement.

The sample analyzed may be any food or beverage. Food and beverage examples are described in Example 6, Table 5. However, the disclosure is not limited to these foods and beverages.

As demonstrated in the examples, the concentration range of Al³⁺ ion sensitivity of the method is from 0.008 to 1070 μM, and the response time of the potentiometric aluminum ion sensor to obtain the Al³⁺ concentration is 6 to 10 seconds.

Aluminum toxicity remains a critical clinical issue, particularly among patients with renal impairment, individuals receiving parenteral nutrition, those with Alzheimer's disease, and children with neurodevelopmental disorders such as autism and ADHD. These vulnerable populations highlight the urgent need for reliable monitoring tools. As described in the bioanalytical validation study described in Example 8, a complete bioanalytical validation of the disclosed potentiometric sensor for the sensitive and specific detection of aluminum in simulated blood plasma was conducted. The study established the sensor's analytical performance in accordance with established bioanalytical method validation principles.

The bioanalytical validation study demonstrated that the developed MWCNT-modified quercetin-based potentiometric sensor meets established performance criteria for electrochemical bioanalytical methods. The sensor exhibits high sensitivity, a broad linear dynamic range covering physiologically and pathophysiologically relevant aluminum concentrations, excellent selectivity against common biological interferents, and outstanding accuracy and precision within a simulated plasma matrix. The validation protocol was performed in accordance with established bioanalytical method validation principles, confirming the sensor's reliability, robustness, and reproducibility for routine aluminum determination in biological systems.

In addition to invasive matrices such as blood, the sensor has been successfully applied to non-invasive matrices, including digested human hair and nail samples, offering an effective approach for monitoring chronic aluminum exposure. These non-invasive biomarkers are particularly valuable in vulnerable populations, such as children with neurodevelopmental disorders (e.g., ASD and ADHD), where long-term exposure assessment is critical for understanding potential environmental and toxicological contributions.

Collectively, these results establish the developed quercetin-based MWCNT sensor as a versatile, selective, and clinically adaptable platform, representing a significant advancement toward point-of-care diagnostics, environmental monitoring, and personalized therapeutic management of aluminum exposure.

Thus, in a fourth embodiment, the present disclosure provides a method for detecting aluminum ion concentration in a biological fluid to be analyzed. According to the method, the pH of the biological fluid is adjusted to 4.5, and a sample of the pH-adjusted material is contacted with the potentiometric aluminum ion sensor previously described as the second embodiment, and a potential emf value is read from the potentiometer. The obtained emf value is compared to a calibration curve of emf value versus log aluminum ion (Al³⁺) in M to determine the Al³⁺ M value of the sample. The pH-adjusted biological fluid may be tested directly without any purification.

According to the method of the fourth embodiment, a concentration range of Al³⁺ ions of the biological fluid is from 0.023-100 μM, and a response time of the potentiometric aluminum ion sensor to obtain the Al³⁺ concentration is from 6 to 10 seconds.

The above description is presented to enable a person skilled in the art to make and use the embodiments and aspects of the disclosure and is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown but is to be accorded with the widest scope consistent with the principles and features disclosed herein. In this regard, certain embodiments within the disclosure may not show every benefit of the disclosure, considered broadly.

### EXAMPLES

Aluminum, manganese, sodium, potassium, calcium, ferric, and lead chloride, cadmium, copper, and nickel sulfate, potassium hydroxide, hydrogen peroxide, nitric acid, hydrochloric acid, potassium Tetrakis 4-chlorophenyl Borate (KTCPB), high molecular-weight Poly(vinyl chloride) (PVC), 2-Nitrophenyl octyl ether (NOPE), multiwalled carbon nanotubes (MWCNTs), 2-methyl tetrahydrofuran (MeTHF), quercetin, morin Hydrate, di-iodohydroxy quinoline, and calix[4] arene (CX4) were purchased from Sigma-Aldrich Chemie GmbH (Steinheim, Germany).

1. Green Extraction of Quercetin from Elderberry Leaves Using Microwave-Assisted Extraction (MAE)

### Procedure:

### 1. Preparation of Plant Material:

The elderberry leaves were thoroughly dried in a drying oven at 57-62° C. for 6-8 hours or until the leaves were completely dry and brittle. The dried leaves were then ground into a fine powder using a mortar and pestle and passed through a 40-mesh sieve. (size: 0.425 mm).

### 2. Microwave-Assisted Extraction (MAE):

10 grams of the powdered plant material were weighed into a 250 mL flask. 100 mL of 95% ethanol was added to the plant material. The mixture was subjected to microwave irradiation at 600 W for 5 minutes at a temperature of 60° C. The mixture was then microwaved, allowing it to cool down to room temperature. The extract was filtered using filter paper to remove plant debris, and the solvent was removed using a rotary evaporator at 40° C. under reduced pressure, yielding a concentrated extract.

### 3. Purification:

The crude extract can be further purified using recrystallization in ethanol. The impure compound was dissolved in 100 ml of hot ethanol. Then, the solvent was heated to its boiling point (78° C.) to ensure complete dissolution, and the hot solution was then filtered to remove insoluble impurities using a fluted filter paper to prevent clogging.

The hot solution was cooled slowly for more than 4 hours in an ice bath to induce crystallization. It was learned that slow cooling allowed for the formation of larger, purer crystals. The crystals were filtered using vacuum filtration overnight and washed with a small amount of cold ethanol to remove any remaining impurities. The crystals were dried thoroughly by using a vacuum desiccator for 6 hours.

Using MAE significantly reduced the extraction time compared to traditional methods. The high temperature and pressure generated by microwaves enhance the mass transfer of compounds from the plant matrix to the solvent.

The quality and purity of the Quercetin may be determined by conventional analytical methods, including ¹H NMR, FTIR, and XRD analysis.

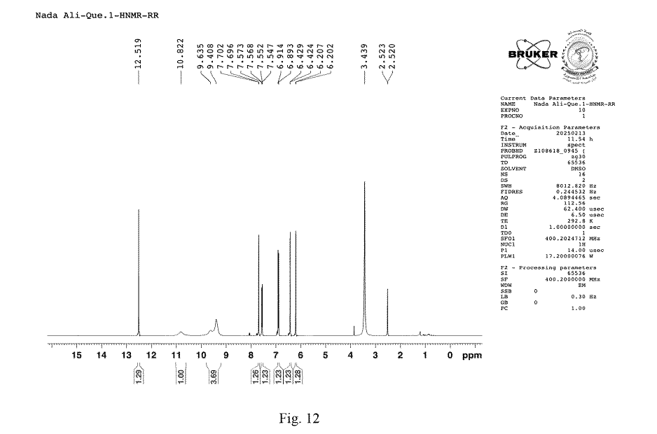

FIGS. 12-16 shows a ¹H NMR spectral analysis of a Quercetin sample obtained employing the following experimental parameters:
Solvent: DMSO-d₆ (suppresses OH exchange but allows observation under low-temperature conditions).
Frequency: 400.20 MHz (standard for structural resolution).
Scans (NS=16): Moderate signal averaging for noise reduction.
Temperature: 254.8 K (−18° C.) enhances the resolution of exchangeable protons.

The HNMR spectrum aligns with quercetin's structure, confirming the presence of aromatic and hydroxyl proton environments. The low-temperature acquisition in DMSO-d₆ enables observing typically broad hydroxyl signals as discrete peaks, providing detailed structural insights. The aromatic proton splitting (doublets at 6.2-7.7 ppm) reflects coupling patterns consistent with the flavonoid skeleton. However, the extreme downfield shifts (15.14 ppm) warrant further validation to exclude artifacts or impurities. This analysis supports the identification of quercetin, with spectral features characteristic of its phenolic and conjugated aromatic system.

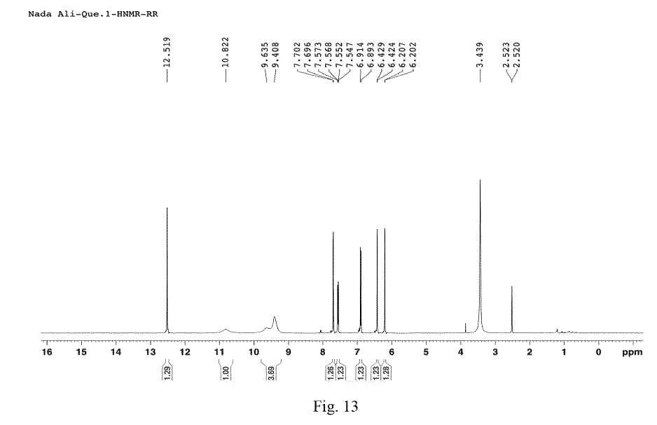

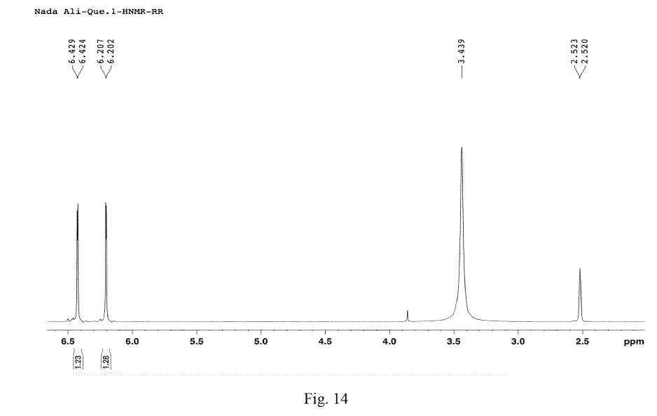

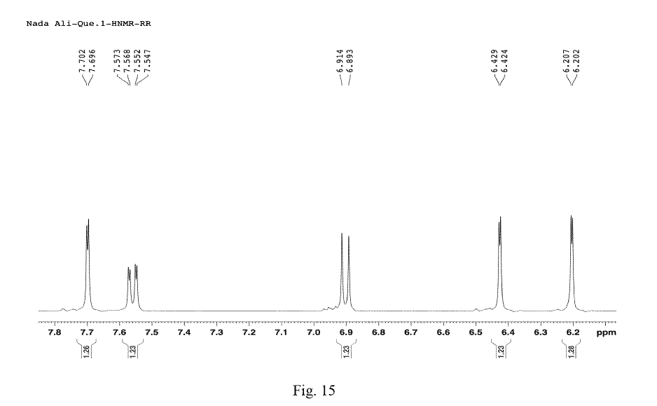

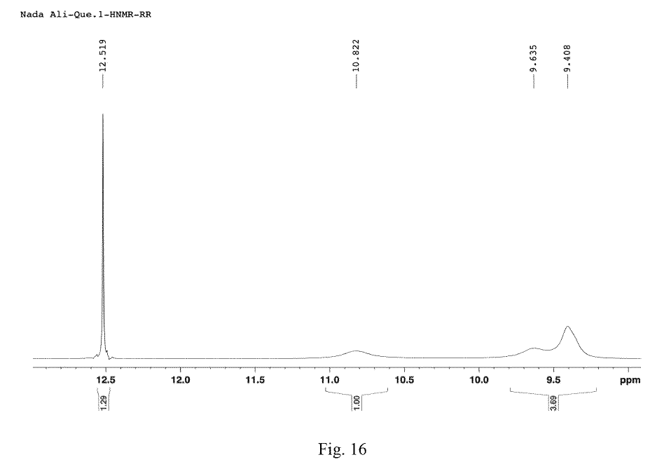

FIGS. 13-16 are each an expansion of a specific region of the quercetin spectrum to analyze coupling patterns or chemical shifts more clearly. FIG. 13 shows an analysis focus on the spectrum of FIG. 12. FIG. 14 shows an expansion of the region from 2.5 ppm to 6.6 ppm of the spectrum of FIG. 12. FIG. 15 shows an expansion of the region from 6.1 ppm to 7.8 ppm of the spectrum of FIG. 12. FIG. 16 shows an expansion of the region from 9.0 ppm to 13 ppm of the spectrum of FIG. 12. Such expansion provides improved peak integration for quantification purposes, and reduces noise and overlapping peaks, making it easier to identify individual protons.

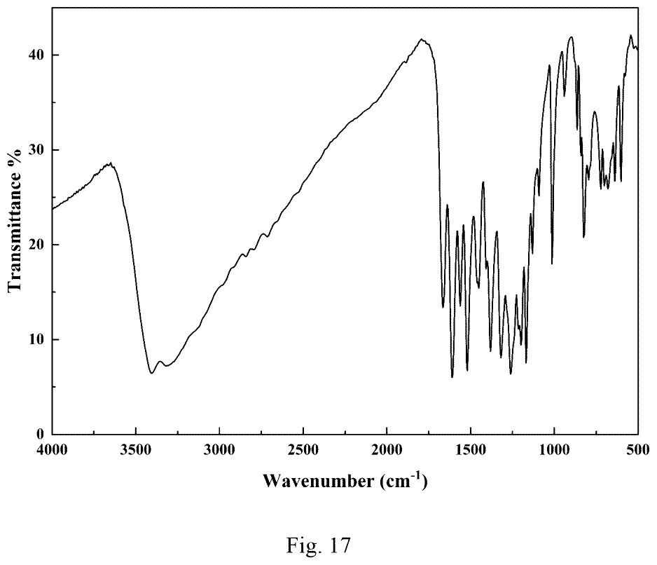

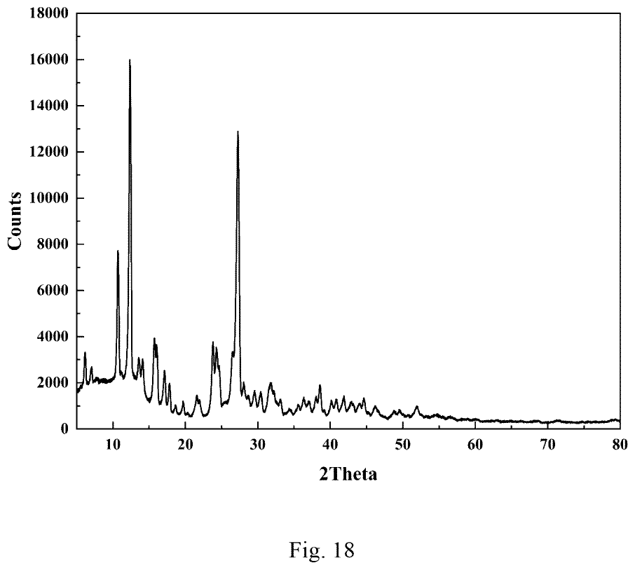

FIG. 17 shows an FTIR spectrum of the quercetin extract sample. The FTIR spectrum reveals characteristic absorption bands corresponding to its functional groups. Key observations include:
1. O—H Stretching (phenolic groups): A broad absorption band in the range of 3500-3000 cm⁻¹ (transmittance ~30-40%) indicates the presence of hydroxyl groups, typical of quercetin's polyphenolic structure.
2. C═O Stretching (carbonyl group): A dip near 1650-1600 cm⁻¹ (likely within the 1500-2000 cm⁻¹ region with transmittance ~10-30%) suggests the carbonyl group in the flavone backbone.
3. Aromatic C═C Stretching: Peaks around 1500-1600 cm⁻¹ (transmittance ~10%) confirm conjugated aromatic ring vibrations.
4. C—O Stretching (ether/phenolic): Bands in the 1200-1300 cm⁻¹ range (within the 1000-1500 cm⁻¹ region) align with C—O bonds in the flavonoid structure.
   The FTIR analysis confirms the presence of quercetin's functional groups, including phenolic-OH, conjugated carbonyl (C═O), and aromatic C═C systems. The spectrum aligns with literature data for quercetin. This analysis supports the successful extraction of quercetin with characteristic structural integrity.
   FIG. 18 shows an XRD analysis of the quercetin extract sample. The presence of distinct, sharp diffraction peaks (at specific 2Theta values, inferred from the intensity variations) suggests a crystalline structure in the quercetin extract.

### 2. Fabrication of Aluminum Liquid-Contact Sensors

### Procedure:

Initial evaluation efforts focused on determining the optimal membrane composition through liquid contact electrode fabrication containing the different ionophores: Quercetin, Morin, Di-iodohydroxyquinoline, and Calixarene 4.

### 1. Membrane Preparation:

Mixing: 33.24% PVC, 66.6% of nitrophenyl octyl ether as a plasticizer, 0.5 mmol/Kg of potassium tetrakis 4-chlorophenyl borate as an ion exchanger, and 1 mmol/Kg of each ionophore, were combined.
Solvent Casting: A quantity of 600 mg of membrane components was solubilized in 6.0 mL of MeTHF.
Casting: The solution was poured into a Petri dish and allowed the solvent to evaporate overnight at room temperature.
Membrane Formation: A thin, flexible membrane was formed. The membrane composition and thickness can be adjusted to optimize the electrode's performance.

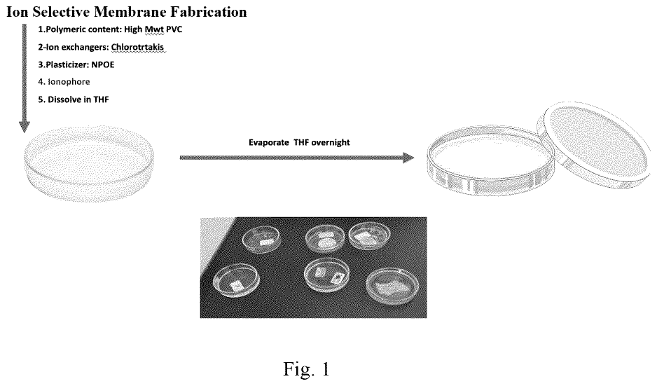

The method to prepare the membrane is shown in FIG. 1.

### 2. Electrode Assembly:

Punching Disks: 5 mm diameter disks were punched from the membrane.
Adhesion: The membrane disk was attached to one end of a PVC tube using MeTHF as an adhesive.
Internal Solution: The tube was filled with 0.1 mM aluminum chloride solution.
Reference Electrode: An Ag/AgCl wire was inserted into the solution as the internal reference electrode.

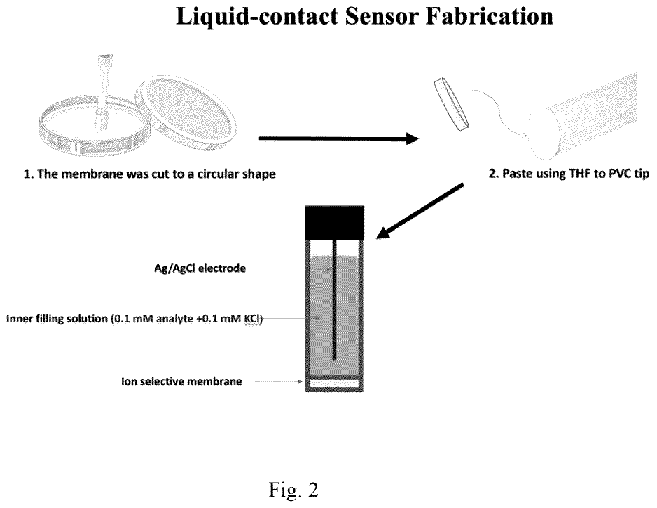

The method to prepare the liquid contact sensors is demonstrated in FIG. 2

### 3. Conditioning:

Soaking: The electrodes were immersed in 0.1 mM aluminum chloride solution for 10 hours at 25° C.
Conditioning: This step allowed the membrane to equilibrate with the target ion and stabilize its response. This conditioning step is crucial for obtaining stable and reproducible measurements.

The membrane compositions are listed in Table 1.

TABLE 1
Membrane compositions of liquid contact membranes

| | | | NOPE | KTCPB Ion |
|---|---|---|---|---|
| | PVC | | Plasticizer | exchanger |
| Sensor | (Wt %) | Ionophore (Wt %) | (Wt %) | (Wt %) |
| 1 | 33.24% | Ionophore free | 66.67% | 0.16% |
| 2 | 33.24% | CX4 | 66.67% | 0.16% |
| 3 | 33.24% | Quercetin | 66.67% | 0.16% |
| 4 | 33.24% | Morin | 66.67% | 0.16% |
| 5 | 33.24% | Di-iodohydroxy | 66.67% | 0.16% |
| | | quinoline | | |

The fabricated liquid-contact sensors were evaluated based on linearity range, slope, response time, and selectivity. The results are shown in Table 2.

TABLE 2
Metrological parameters for liquid contact sensors

| | | Sensor 1 | | | | SensorO5 |
|---|---|---|---|---|---|---|
| | | (ionophore | | Sensor 3 | | (diiodohydroxy |
| | | free) | Sensor 2 (CX4) | (Quercetin) | Sensor 4 (Morin) | quinoline) |
| Slope | | 17.9 ± 3.4 | 20.5 ± 1.7 | 19.0 ± 0.7 | 19.5 ± 0.8 | 19.4 ± 1.3 |
| Range (μM) | | 10-10⁴ | 10-10⁴ | 1-10⁴ | 10-10⁴ | 10-10³ |
| R | | 0.99875 | 0.9975 | 0.9991 | 0.9993 | 0.9992 |
| Response time | | 20 ± 5 | 10 ± 5 | 3 ± 2 | 12 ± 3 | 12 ± 4 |
| Selectivity | Cu (II) | −0.8 | −1.4 | −2.4 | −1.7 | −1.2 |
| toward | Pb (II) | −0.7 | −0.9 | −3.5 | −2.1 | −1.4 |
| Al(III) | Mn (II) | −0.8 | −1.1 | −2.5 | −1.9 | −1.7 |
| | Cd(II) | −1.3 | −1.6 | −3.4 | −3.1 | −2.7 |
| | Fe (III) | −0.7 | −0.9 | −1.9 | −1.6 | −1.4 |

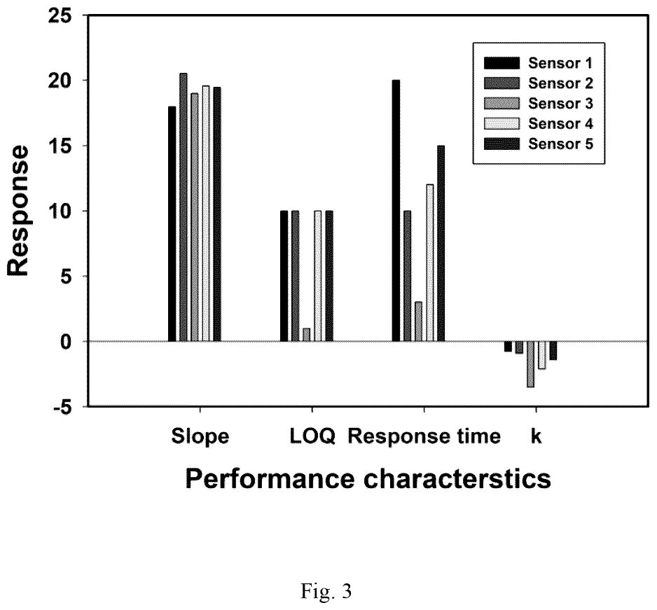

Other performance data collected for the liquid contact sensors listed in Table 2 is shown in FIG. 3.

### 3. Fabrication of Screen-Printed Solid Contact Electrodes (SPEs)

### 1. Substrate Preparation:

Plastic substrates were selected.
The substrate was cleaned ultrasonically in acetone, followed by deionized water to remove any impurities.
The cleaned substrate was dried in an oven at 80° C. for 30 minutes.

### 2. Ink Preparation:

Conductive Ink: A carbon-based ink containing graphite powder (60-80% by weight of the total ink composition) and an acrylic polymer binder (20-30% by weight) was prepared. The ink was ball-milled to ensure uniform particle size distribution.
Reference Electrode Ink: A silver chloride-based ink was prepared by mixing silver chloride powder (50-70% by weight) with ethanol (Enough to dissolve the silver chloride) and an acrylic polymer binder (20-30% by weight).

### 3. Screen Printing Process:

A stainless steel mesh screen with the desired electrode pattern was used for printing.
The conductive ink was applied to the substrate using a squeegee to create the working electrode.
The reference electrode ink was applied to the substrate, ensuring electrical isolation from the working electrode.

### 4. Drying:

The printed electrodes were dried in an oven at 120° C. for 1 hour to ensure complete solvent evaporation and curing of the ink.

### 5. Post-Processing:

Thermal Treatment: the electrodes were annealed at 300° C. for 2 hours in a nitrogen atmosphere to improve electrical conductivity and mechanical stability.
Chemical Modification: the electrode surface was then modified with the ion-selective membrane containing quercetin as ionophore, NOPE as plasticizer, potassium tetrakis 4-chlorophenyl borate as ion exchanger, PVC as polymer binder, and MWCNT transducer.

### 6. Electrical Connections:

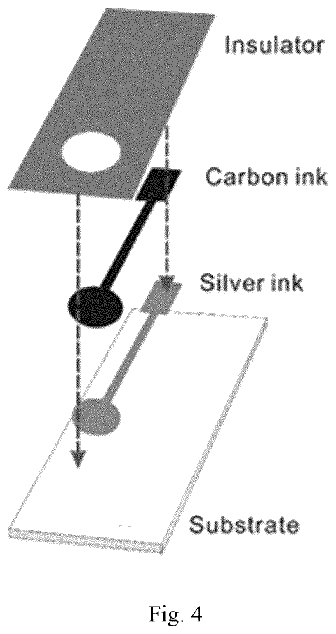

Electrical wires were attached to the working and reference electrodes using conductive adhesives, and we started our measuring processes.
FIG. 4 shows a schematic drawing of the fabrication process.
   4. Performance Comparison of Solid Contact Sensors with and without MWCNTs
Screen-printed electrodes were used for the fabrication of the solid contact electrodes. These SPEs with dimensions of 50×13 mm, feature a 3 mm diameter central disk as a carbon working electrode, with an outer annular carbon auxiliary crescent, and Ag/AgCl pellet reference electrode.

### 1. Membrane Preparation:

A liquid mixture was prepared of the ion-selective membrane components (Ionophore: quercetin, Plasticizer: NOPE, Ion exchanger: potassium tetrakis 4-chlorophenyl borate, and Polymer:PVC).

### 2 Drop-Casting:

The liquid membrane mixture was directly applied onto the desired surface of the screen-printed electrode.

### 3. Solvent Evaporation:

The solvent was allowed to evaporate, leaving a solid membrane layer on the electrode surface.

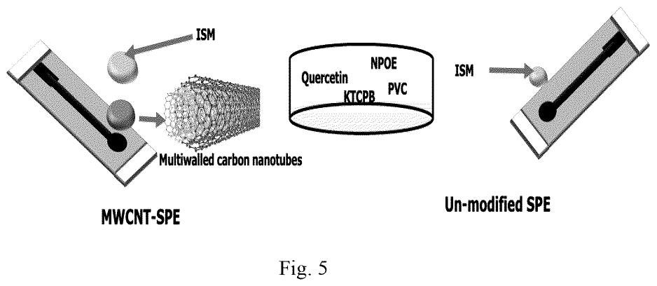

A sensor was prepared as outlined above and further modified as follows
MWCNT Dispersion:
MWCNTs Specifications
Appearance: Black Powder
Diameter 50-90 nm
Ratio <0.15_D/G
Carbon Content >95.0%
10 mg of MWCNTs were dispersed in 10 mL of MeTHF using sonication for 20 minutes.
Membrane-MWCNT Mixture:
The ion-selective membrane solution was mixed with the MWCNT suspension in equal volumes, and the mixture was sonicated for 10 minutes to ensure uniform dispersion.
Drop-Casting:
A 20 μL of the MWCNT-modified membrane mixture was applied onto the electrode surface, and the mixture was allowed to dry overnight.
FIG. 5 shows a comparative schematic picture of the method to prepare the unmodified screen-printed electrode (SPE) and the modified SPE.
FIG. 11 shows a scale drawing of a sensor according to one embodiment as prepared.

The Metrological parameters (regression and validation data) of the unmodified (no MWCNTs) and modified microfabricated sensors for aluminum determination are provided in Table 3.

TABLE 3

| Parameter | Modified | Unmodified |
|---|---|---|
| Slope (m V/decade) ± SD | 21.9 ± 0.4 | 22.3 ± 0.7 |
| Intercept (mV) ± SD | 188.6 ± 0.5 | 143.62 ± 0.9 |
| LOD (μM) | 0.008 | 0.91 |
| Response time (s) | 3 ± 1 | 7 ± 2 |
| Linear range (μM) | 0.023-1070 | 1.07-1070 |
| Correlation coefficient | 0.9996 | 0.9987 |
| Accuracy ± SD | 101.9 ± 0.51 | 97.9 ± 1.30 |
| Repeatability | 0.82 | 0.91 |
| Intermediate precision | 0.90 | 1.37 |

The above metrological parameters correspond to aqueous calibration conditions. Matrix-dependent variations in analytical performance are described separately in Example 8 for simulated plasma

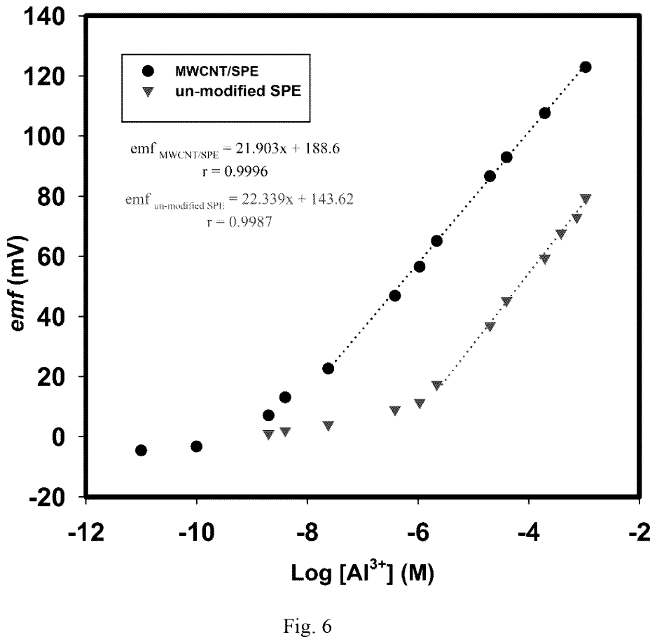

FIG. 6 shows a profile of the measured potential in mV versus log concentrations of aluminum in M obtained with the MWCNT/ISM and MWCNT-free sensor. The emf values were recorded in a solution with a pH of 4.5 at room temperature.

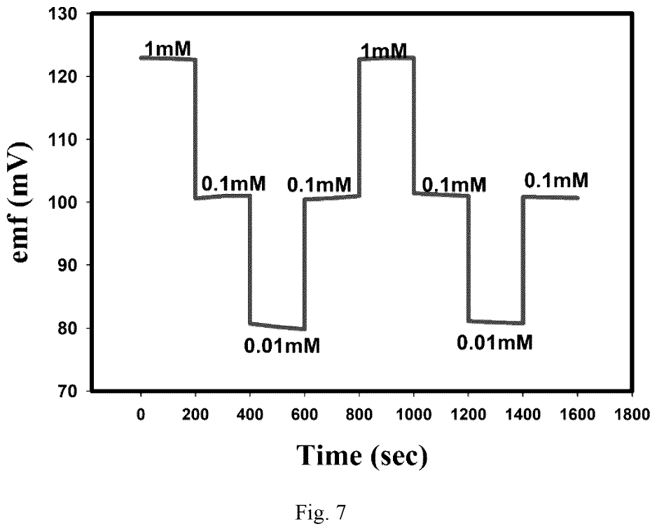

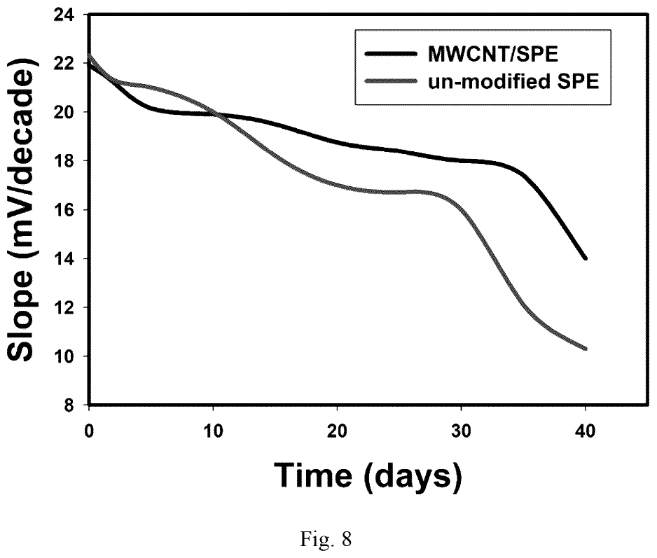

The reversibility of the MWCNT modified sensor, observed as Al³⁺ concentrations alternate between indicated values, is shown in FIG. 7, and the comparison of long-term stability of the modified and unmodified sensors is shown in FIG. 8.

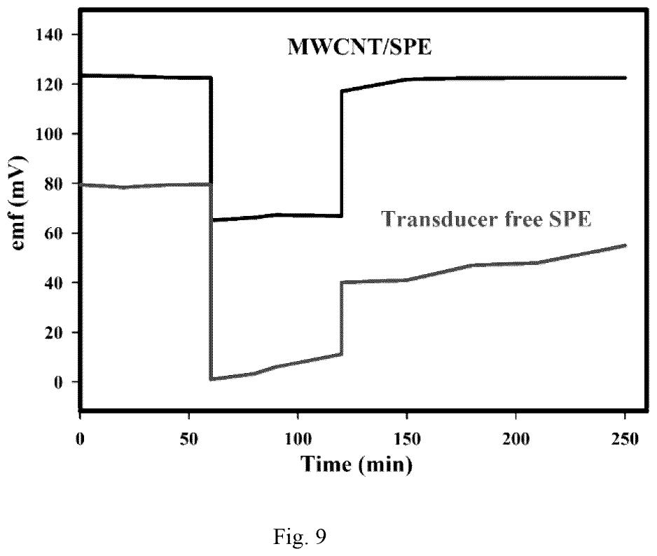

FIG. 9 shows the results of water layer testing of the MWCNT-modified sensor and MWCNT free sensor. Potential drift is recorded when using the MWCNTs free sensor, indicating the accumulation of the water layer between the sensing membrane and the substrate.

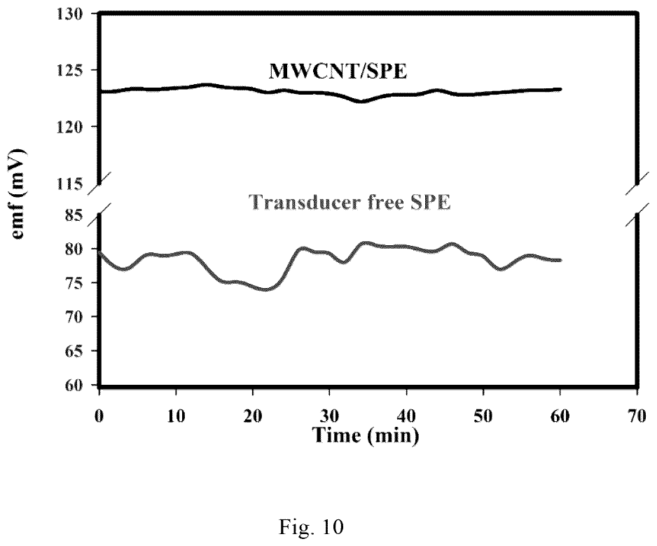

FIG. 10 shows the short-term potential stability of the MWCNT microfabricated sensor and MWCNT-free one upon measuring in a 0.10 mM aluminum solution for one hr. (B). In the presence of the MWCNT layer, the potential drift was reduced from 0.75 mV/h to 0.024 mV/h.

### 5. Determination of the Selectivity of the MWCNT-Modified Sensor Towards Aluminum Ions.

The selectivity of the MWCNT-modified sensor towards aluminum ions was evaluated using the separate solutions method. The sensor's performance was assessed in the presence of various potential interfering ions, such as copper, lead, potassium, manganese, cadmium, sodium, zinc, nickel, cobalt, calcium, iron, and other cations commonly found in food and beverage samples. Logarithmic selectivity coefficients (log K) were calculated to quantify the sensor's selectivity for aluminum ions over other ions. The results demonstrated that the sensor exhibited excellent selectivity for aluminum ions with minimal interference from other cations. The strong binding affinity between aluminum ions and the quercetin ionophore played a crucial role in achieving high selectivity. The strong interaction between the target ion and the ionophore facilitates the formation of a highly selective complex, leading to enhanced selectivity for aluminum ions. Table 4 shows the selectivity coefficient of the MWCNT modified sensor for Aluminum over potentially interfering ions I

$$( Log {K}_{Al .}^{pot} ) .$$

Each value shown is the average of three determinations.

TABLE 4

| | $( Log {K}_{Al ( III ) . I}^{p o t} )$ |
|---|---|
| Interferent | MWCNT−SPE |
| Cu (II) | −2.42 |
| Pb (II) | −3.39 |
| Mn (II) | −1.96 |
| Cd (II) | −3.37 |
| Ni (II) | −3.00 |
| Na (I) | −1.95 |
| Fe (III) | −2.10 |
| Ca (II) | −2.71 |
| K (I) | −1.87 |

### 6. Validation of Aluminum Determination Using Standard MP-AES Method

Microwave Plasma Atomic Emission Spectroscopy (MP-AES) was used as a reference technique to validate the accuracy of the sensor's results.
MP-AES Parameter Optimization:
Wavelength: 396.152 nm
Pump Speed: 15 rpm
Nebulizer Flow Rate: 0.95 L/min
Uptake Time: 25 seconds
Stabilization Time: 25 seconds (repeated three times)

The aluminum ion concentration obtained using the fabricated MWCNT modified screen-printed sensor (MWCNT/SPE) was compared to the results obtained using MP-AES for various food and beverage samples. A variety of food and beverage samples were selected, including fresh produce, processed foods, and beverages, and prepared for analysis as indicated in Table 5. The tested foodstuffs (fresh tomato, tomato sauce, cheese, soft drinks, cooking oil, potatoes, spinach, black pepper, paprika, salt, cocoa powder, ready-made cake powder, rice, tea leaves, and fried liver and brain) were purchased from local stores and markets in Egypt. Table 6 lists the comparative aluminum concentration in ppm obtained with MP-AES and the MWCNT/SPE.

TABLE 5

| Sample type | Preparation Method | Digestion Conditions |
|---|---|---|
| Fresh | Washed, sliced, | 5 mL 69% HNO3 + 2 mL |
| Vegetables (tomato, | weighed, and transferred to | 30% H2O2, 145° C. for 5 min, |
| potato, spinach) | digestion vessel | 170° C. for 5 min, 190° C. for 15 |
| | | min, 75° C. for 10 min |
| Tomato Sauce | Homogenized, weighed, | 5 mL 69% HNO3 + 2 mL |
| | prepared in an aluminum pot | 30% H2O2, 145° C. for 5 min, |
| | and cooked for 30 minutes, and | 170° C. for 5 min, 190° C. for 15 |
| | transferred to digestion vessel | min, 75° C. for 10 min |
| Liver and Brain | Homogenized, weighed, | 5 mL 69% HNO3 + 2 mL |
| | and transferred to digestion | 30% H2O2, 145° C. for 5 min, |
| | vessel | 170° C. for 5 min, 190° C. for 15 |
| | | min, 75° C. for 10 min |
| Salt | Weighed and transferred | 10 mL 69% HNO3 + 2 mL |
| | to digestion vessel | 30% H2O2, 135° C. for 5 min, |
| | | 150° C. for 5 min, 190° C. for 15 |
| | | min, 75° C. for 10 min |
| Pepper, Paprika, | Weighed and transferred | 5 mL 69% HNO3 + 2 mL |
| Cocoa Powder, Ready- | to digestion vessel | 30% H2O2, 150° C. for 5 min, |
| made Cake Mix | | 200° C. for 10 min (twice), 75° C. |
| | | for 10 min |
| Rice (dry, | Weighed and transferred | 10 mL 69% HNO3 + 2 mL |
| water-soaked, cooked) | to digestion vessel | 30% H2O2, 160° C. for 5 min, |
| | | 160° C. for 10 min, 190° C. for 10 |
| | | min, 75° C. for 10 min |

TABLE 6

| | |
|---|---|
| Food or beverage | Aluminum concentration (ppm) |

| Sample | MP-AES | MWCNT/SPE |
|---|---|---|
| Black pepper 1 | 10.42 | 9.7 |
| Black pepper 2 | 78.58 | 77.41 |
| Black tea 1 | 43.23 | 40.9 |
| Black tea 2 | 78.94 | 75.10 |
| Black tea (raspberry) 3 | 74.88 | 73.90 |
| Cocoa powder 1 | 14.35 | 12.2 |
| Cocoa powder 2 | 3.19 | 2.60 |
| Cocoa powder 3 | 3.19 | 2.88 |
| Paprika powder 1 | 23.23 | 22.9 |
| Paprika powder 2 | 12.47 | 11.76 |
| Paprika powder 3 | 12.44 | 12.0 |
| Chocolate cake | 2.19 | 1.51 |
| Creamy cheese | 2.37 | 1.8 |
| Triangle cheese | 2.42 | 1.7 |
| Rice 1 | 3.0 | 2.7 |
| Coca-Cola | 39.82 | 40.51 |
| Potatoes | 4.04 | 4.67 |
| Tomato sauce 1 | 9.91 | 10.3 |
| Tomato sauce 2 | 2.02 | 1.7 |

### 7. Determination of Binding Energy Between Al³⁺ and Studied Ionophores

To validate the experimental results of Example 2, the specific preference of quercetin for aluminum ions and their strong bonding interactions was compared to the other ionophores studied by calculating binding energies using DFT calculations as outlined below. The computational protocol used in this study has been successfully applied to similar systems.
Software: Gaussian 09 software package
Functional: B3LYP (Becke three-parameter Lee-Yang-Parr hybrid exchange-correlation functional)
Basis Set: 6-311++G(d,p)

### Optimization and Analysis:

Geometry Optimization: All ionophore structures and their aluminum complexes were fully optimized.
Vibrational Frequency Calculations: Harmonic vibrational frequencies were calculated to confirm the optimized structures as true minima.
Thermochemical Analysis: Thermochemical analysis was performed to obtain Gibbs free energies.
Solvent Effects: The impact of the solvent (2-nitrophenyl octyl ether) was simulated using the implicit polarizable continuum model (PCM) with 1-nitropropane as a solvent surrogate.

### Initial Optimization:

The quercetin molecule was initially optimized to establish a baseline for subsequent complex calculations.
Quercetin's Coordination Sites: Quercetin, a polydentate ligand, can coordinate with metal ions through multiple sites:
   3-hydroxyl and 4-carbonyl groups on the C ring (3-4 site)
   4-carbonyl and 5-hydroxyl groups on both A and C rings (4-5 site)
   Catechol moiety on the B ring (3′-4′ site)
Metal-Ligand Interaction: The specific binding site and metal-ligand stoichiometry can vary depending on factors like metal ions, pH, and solvent polarity.
Aluminum-Quercetin Complex: For aluminum, a deprotonated form of quercetin was considered, forming a 1:1 metal-ligand complex.
Strong Binding Affinity: The strong binding affinity between aluminum ions and quercetin was attributed to the electrostatic interaction between the positively charged metal ion and the negatively charged deprotonated ligand.
Planarity and Conjugation: The planarity of quercetin enhances its conjugation, which plays a role in its antioxidant properties and metal-binding ability.
Comparison with Other Ligands: Quercetin exhibited a higher binding affinity for aluminum ions compared to other ligands like morin and di-iodohydroxy quinoline. This supports the experimental findings and highlights quercetin's unique suitability for aluminum ion sensing.

The calculated results are shown in Table 7.

TABLE 7

| | | Thermodynamic | |
|---|---|---|---|
| Quercetin | Electronic Energy (E) | correction Factor | ${G}_{AB = {G}_{A +} {G}_{B}} {=}^{Δ} G$ |
| Al | −241.3278725 | −0.014655 | −384.986 |
| Ligand | −1104.174944 | 0.168438 | |
| Complex | −1346.135924 | 0.173376 | |
| | | Thermodynamic | |
| Morin | Electronic Energy (E) | correction Factor | ${G}_{AB = {G}_{A +} {G}_{B}} {=}^{Δ} G$ |
| Al | −241.3278725 | −0.014655 | −384.482 |
| Ligand | −1104.175067 | 0.169488 | |
| Complex | −1346.134418 | 0.173601 | |
| | | Thermodynamic | |
| Diiodohydroxy quinoline | Electronic Energy (E) | correction Factor | ${G}_{AB = {G}_{A +} {G}_{B}} {=}^{Δ} G$ |
| Al | −241.3278725 | −0.014655 | −370.184 |
| Ligand | −1071.25925 | 0.064305 | |
| Complex | −1313.195627 | 0.068229 | |

### 8. Bioanalytical Validation of Detection of Aluminum in Simulated Blood Plasma Chemicals and Instrumentations

Aluminum, sodium, potassium, calcium, ferric, and magnesium chloride, copper sulfate, potassium hydroxide, hydrogen peroxide, nitric acid, hydrochloric acid, Potassium tetrakis(4-chlorophenyl)borate (KTpCIPB), high molecular-weight Poly(vinyl chloride) (PVC), 2-Nitrophenyl octyl ether (NOPE), multiwalled carbon nanotubes (MWCNTs), 2-methyl tetrahydrofuran (MeTHF) were purchased from Sigma-Aldrich Chemie GmbH (Steinheim, Germany). Elderberry leaves were purchased from an Egyptian farm. Simulated plasma fluid was obtained from Biochemazone (Alberta, Canada). Potentiometric measurements were carried out using a 6-channel potentiometer (Lawson Labs, USA) and a double-junction Ag/AgCl reference electrode (Thermo Scientific Orion, USA). Microwave Plasma-Atomic Emission Spectroscopy (MP-AES) was used as the reference analytical method. All glassware was cleaned with distilled water and oven-dried before use.

### Preparation of Metal Standard Solutions in Simulated Plasma

A 1.0 mM Al³⁺ stock solution was prepared in ultra-pure water and used to generate calibration standards by spiking simulated plasma (Biochemazone, Canada). The resulting concentrations ranged from 1.0×10⁻⁸ M to 1.0×10⁻³ M, encompassing physiologically relevant levels of aluminum. Calibration curves were constructed by recording the emf values of successive plasma dilutions prepared from the stock solution.

For the selectivity study, simulated plasma samples were similarly spiked with 0.1 mM of potential interfering ions (Na⁺, K⁺, Ca²⁺, Mg²⁺, Fe³⁺, Cu²⁺). The selectivity coefficients were then determined in the plasma matrix under the same conditions as calibration to ensure matrix relevance and prevent Al³⁺ hydrolysis under near-neutral conditions.

### Analysis of Simulated Plasma Samples

Simulated plasma was employed as the validation matrix. For the recovery study, samples were spiked with aluminum at three levels within the calibration range: Low (2.5×10⁻⁸ M), Medium (5.0×10⁻⁶ M), and High (5.0×10⁻⁵ M). Each was analyzed in sextuplet (n=6) across three different days to evaluate intra- and inter-day accuracy and precision. No digestion was required before potentiometric analysis.

Potentiometric measurements were performed at room temperature (~25° C.) with constant stirring, using a high-impedance (10¹³Ω) 6-channel potentiometer connected to the quercetin-based ion-selective electrode and a double-junction reference electrode (3.0 M KCl saturated with AgCl as the inner filling solution, 0.1 M KNO₃ as the bridge electrolyte to avoid chloride leakage). Both electrodes were immersed directly into the plasma samples, and the potential was recorded after the samples had stabilized.

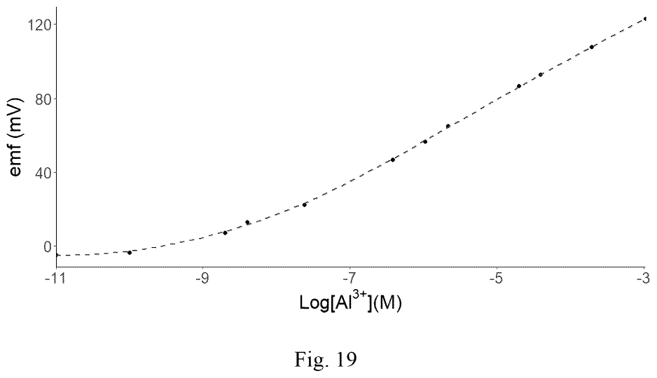

Calibration was performed using spiked simulated plasma standards spanning 1.0×10⁻⁸ to 1.0×10⁻³ M Al³⁺ at pH 4.5±0.1. FIG. 19 shows a calibration curve prepared. The calibration curve was constructed by plotting the electrode potential against the logarithm of the aluminum concentration. Method performance was validated in accordance with IUPAC guidelines.

It is important to note that while simulated plasma was originally at pH 7.4±0.1 to reflect physiological conditions, calibration, and potentiometric measurements were optimized at pH 4.5±0.1. This adjustment was essential to maintain aluminum solubility and ensure electrode stability, as near-neutral pH promotes aluminum hydrolysis and precipitation, which would impair sensor performance.

### Aluminum Determination Using MP-AES and the Proposed Potentiometric Sensors

This study utilized MP-AES as a reference method to validate our proposed sensor accuracy, compare detection limits, and identify potential discrepancies. For aluminium quantification, measurements were performed in triplicate, and the mean values were reported. The MP-AES parameters were optimized for aluminium and were set as follows: wavelength at 396.152 nm, pump speed at 15 rpm, nebulizer flow rate at 0.95 L/min, uptake, and stabilization times at 25 seconds each. Unlike real plasma, the simulated plasma used in this study did not require digestion before MP-AES analysis.

For potentiometric measurements, the membrane sensor and reference electrode were immersed in each sample solution, with thorough water rinsing between measurements. Aluminum concentrations were determined from recorded emf values using the Nernst equation calibration for the proposed sensors.

### Results and Discussion: Bioanalytical Validation in Simulated Plasma Comprehensive Performance Validation

The invented sensor was rigorously validated to establish its performance for quantifying aluminum (Al³⁺) in a simulated plasma matrix, demonstrating its suitability for clinical monitoring applications across both normal physiological and toxicological concentration ranges.

### Sensitivity and Linearity

The MWCNT/SPE sensor demonstrated exceptional analytical performance across a clinically relevant concentration range of 0.023-100 μM (2.3×10⁻⁸-1.0×10⁻⁴ M). This range was strategically selected to encompass the entire spectrum of physiologically significant aluminum levels, from the sub-baseline concentration of 0.023 μM to levels indicative of severe toxicity (100 μM). Specifically, the sensor's operational window was demonstrated to cover:
Normal physiological levels: ~0.037-0.11 μM (≈1-3 μg·L⁻¹) found in healthy individuals.
Clinically concerning concentrations: 0.74-2.22 μM (≈20-60 μg·L⁻¹)—critical for monitoring dialysis patients and individuals with renal impairment.
Toxicological thresholds: >3.7 μM (≈>100 μg·L⁻¹) associated with aluminum overload and toxicity.

The sensor exhibited a well-defined linear potentiometric response characterized by a near-Nernstian slope of 20.02±0.4 mV decade⁻¹ with excellent correlation (r=0.9928−0.9996). The remarkable limit of detection (LOD) of 8.0×10⁻⁹ M (0.008 μM) confirms the sensor's capability to detect aluminum concentrations even below normal physiological baselines. Combined with a rapid response time (6±1 s) and sustained stability, these metrological parameters establish the sensor's suitability for quantitative assessment of both trace and elevated aluminum concentrations in complex biological matrices.

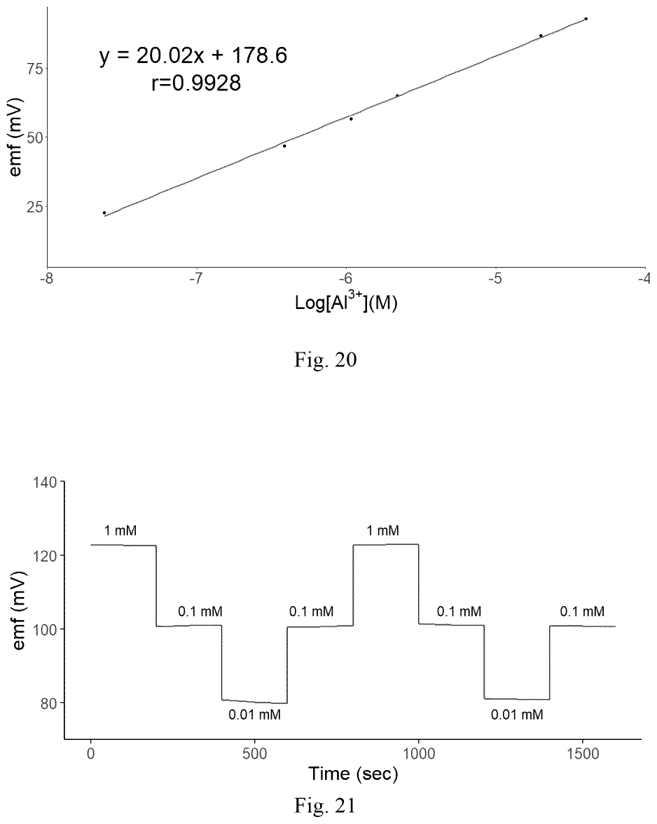

FIG. 20 shows a profile of the measured potential in mV versus log concentrations of aluminum in M obtained with MWCNT/ISM. The EMF values were recorded in a solution with a pH of 4.5 at room temperature.

Note: Calibration standards (1×10⁻⁸ M-1×10⁻³ M) were prepared, although the analytically validated linear range of the sensor extended from 2.3×10⁻⁸ M to 1×10⁻⁴ M.

In simulated plasma, the sensor exhibits a validated linear dynamic range from 2.3×10⁻⁸ M to 1.0×10⁻⁴ M (0.023-100 μM). Although calibration standards were prepared up to 1.0×10⁻³ M, concentrations above 1.0×10⁻⁴ M were considered outside the analytically validated linear region for the plasma matrix.

Table 8 shows the determined metrological parameters of the MWCNT-based sensor in simulated plasma.

TABLE 8

| | |
|---|---|
| Parameter | MWCNT-Modified Sensor (MWCNT/SPE) |
| Linear range | 0.023-100 μM (2.3 × 10⁻⁸-1.0 × 10⁻⁴ M) |
| Slope (mV decade⁻¹) ± SD | 20.02 ± 0.4 |
| LOD⁰ | 8.0 × 10⁻⁹ M (0.008 μM) |
| Response time (s) | 6 ± 1 |
| Correlation coefficient (r) | 0.9928-0.9996 |
| Intercept (mV) ± SD | 178.6 ± 0.5 |
| Accuracy ± SD | 101.8 ± 0.51 |
| Repeatability (RSD %) | 0.79 |
| Intermediate precision | 0.86 |
| (RSD %) | |

^{a} Average of three determinations.
^{b} Limit of detection (measured by interception of the extrapolated arms of nonresponsive and Nernstian segments of the calibration plot of FIG. 2

The observed differences in slope, response time, and validated linear range between aqueous and simulated plasma measurements are attributed to matrix effects, including increased ionic strength, protein interactions, and diffusion limitations, which influence the effective activity of free Al³⁺ ions.

### Accuracy and Precision Across the Extended Concentration Range

Method validation was performed according to established bioanalytical guidelines, with accuracy and precision assessed at five quality control levels spanning the calibration range from the lower limit of quantification (LOQ, 0.023 μM) to the upper limit of the linear range. Both intra-day (n=6) and inter-day (three consecutive days) evaluations demonstrated excellent reproducibility and quantitative recovery, with mean recoveries of 98.4-102.2% and RSD values consistently below 3%. These results confirm that the sensor meets rigorous validation criteria for electrochemical bioassays across all clinically significant aluminum concentrations. Table 9 shows the determined Accuracy and Precision Data for Al³⁺ in Spiked Simulated Plasma.

TABLE 9

| Spiked | | Mean | Recovery | Intra-day | Inter-day |
|---|---|---|---|---|---|
| Concentration (M) | Clinical Context | Found (M) | (%) | RSD (%) | RSD (%) |
| 2.5 × 10⁻⁸ | Below normal | 2.46 × 10⁻⁸ | 98.5 | 1.2 | 1.8 |
| | baseline | | | | |
| 5.0 × 10⁻⁷ | Elevated | 5.09 × 10⁻⁷ | 101.8 | 0.9 | 1.5 |
| | exposure | | | | |
| 5.0 × 10⁻⁶ | Early toxicity | 5.03 × 10⁻⁶ | 100.6 | 1.1 | 1.7 |
| | threshold | | | | |
| 5.0 × 10⁻⁵ | Moderate to | 4.92 × 10⁻⁵ | 98.4 | 1.8 | 2.3 |
| | severe toxicity | | | | |

### Response Time and Reversibility

The sensor demonstrated a rapid response time, achieving stable potential readings (±1 mV of final value) within 6±1 s at Al³⁺ concentrations above 2.0 μM. At lower concentrations approaching the detection limit, response times extended to approximately 15 s, reflecting the expected kinetic behavior in trace-level detection.

Reversibility was rigorously evaluated by cycling the sensor between high-concentration Al³⁺ solutions (1.0×10⁻⁴ M and 1.0×10⁻³ M) to assess potential memory effects and sensor recovery under demanding conditions beyond the upper linear range limit. The sensor exhibited excellent reversibility with no statistically significant hysteresis or signal carry-over. Transition from high-to-low concentration stabilized within 3-10 s, while low-to-high transition achieved equilibrium more rapidly, confirming robust signal recovery and minimal membrane fouling under extreme concentration changes. FIG. 21 demonstrates the performance obtained.

### Long-Term Stability and Reproducibility

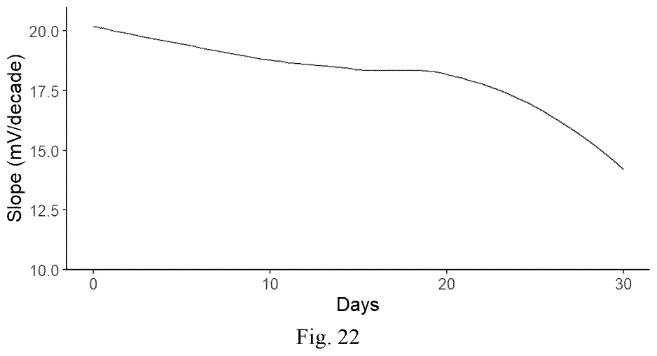

Long-term stability studies revealed consistent potentiometric performance for over 30 days, with minimal slope degradation (≤0.1 mV decade⁻¹ day⁻¹). Batch-to-batch reproducibility was validated using three independently fabricated electrode batches, yielding mean slope and intercept values of 20.07±0.5 mV decade⁻¹ and 178.6±0.5 mV, respectively. The remarkably low variance in both parameters demonstrates exceptional manufacturing consistency, supporting the practical implementation of single-point calibration protocols across sensor production lots. FIG. 22 demonstrates the stability performance obtained.

### Effect of pH

The EMF response to both 0.1 mM and 1.0 mM Al³⁺ was evaluated over a pH range of 1 to 10, and the results are shown in FIG. 23. As indicated, a stable and reproducible response window was identified within this range. Values between 3.0 and 6.0 were noted as optimal, and pH 4.5±0.1 was selected as the preferred optimal working condition, providing maximum signal stability while avoiding H₃O⁺ interference at lower pH values and preventing Al(OH)₃ precipitation at higher pH.

### Selectivity Study

A selectivity study against major physiological cations (Na⁺, K⁺, Ca²⁺, Mg²⁺) and potential interferents (Fe³⁺, Cu²⁺) confirmed excellent selectivity for Al³⁺ across a wide concentration range. Logarithmic selectivity coefficients, log K values, were derived from the calibration curves shown in Table 10. The data clearly highlight the outstanding selectivity of MWCNT/SPEs for aluminum ions, with minimal interference from other ions, likely due to the incorporation of quercetin as an ionophore, given its strong affinity for Al³⁺. A higher binding affinity between the ionophore and the target ion reduces the free energy barrier for transfer from solution to the membrane, thereby selectively enriching the target analyte in the hydrophobic phase.

TABLE 10

| Interfering ion | Log KAl3+, Ipot_{Al³⁺, I}\hat{ }{pot} |
|---|---|
| Na⁺ | −1.95 |
| K⁺ | −1.87 |
| Ca²⁺ | −2.71 |
| Mg²⁺ | −2.20 |
| Fe³⁺ | −2.10 |
| Cu²⁺ | −2.42 |

^{a} Each value is the average of three determinations.

### Aluminum Determination in Spiked Simulated Plasma Using MP-AES and Potentiometric Sensors

The accuracy and applicability of the proposed MWCNT-modified potentiometric sensor were evaluated using spiked simulated plasma samples and benchmarked against microwave plasma-atomic emission spectroscopy (MP-AES) as the reference method. Aluminum was spiked across an extended clinically relevant range (1.0×10⁻⁸ M-1.0×10⁻⁴ M, encompassing ultra-trace, normal, elevated, and toxicological levels. Potentiometric measurements were performed in parallel with MP-AES analysis under identical experimental conditions. Aluminum concentrations were calculated from EMF readings using the established Nernstian calibration curve.

Comparative results (Table 11) demonstrated excellent agreement between both methods across all spiking levels, with no statistically significant differences (p>0.05, paired t-test). The mean recoveries obtained using the potentiometric sensor ranged from 96.2% to 103.1%, closely matching those obtained by MP-AES. The relative standard deviations (RSDs) were ≤4.2% for the potentiometric sensor and ≤3.1% for MP-AES, confirming acceptable precision and reproducibility for both analytical techniques.

Overall, the results validate that the proposed MWCNT-modified potentiometric sensor provides a reliable, reproducible, and highly sensitive approach for aluminum quantification in complex biological matrices. While MP-AES achieved a marginally lower instrumental detection limit, the potentiometric sensor offers significant advantages in simplicity, cost-effectiveness, miniaturization potential, and suitability for continuous or on-site monitoring in physiological and toxicological investigations.

TABLE 11

| | MP-AES ± | MWCNT/SPE ± |
|---|---|---|
| Sample (Spiked Level) | SD (μg/L) | SD (μg/L) |
| Ultra-low (1.0 × 10⁻⁸ M; 0.27 μg/L) | 0.28 | 0.27 |
| Low (2.5 × 10⁻⁷ M; 6.8 μg/L) | 6.9 | 6.7 |
| Medium (5.0 × 10⁻⁶ M; 135 μg/L) | 134.6 | 132.8 |
| High (5.0 × 10⁻⁵ M; 1350 μg/L) | 1348 | 1339 |
| Toxicological (1.0 × 10⁻⁴ M; 2700 | 2703 | 2687 |
| μg/L) | | |

a n = 3, and ±SD denotes the standard deviation.

1. A solid-contact Al ion-selective electrode, comprising:
a substrate;
a layer of an electrically conductive carbon material applied to a surface of the substrate;
an ion-selective membrane in contact with the layer of electrically conductive carbon material, the ion-selective membrane comprising:
quercetin;
multi-walled carbon nanotubes (MWCNT's);
a plasticizer;
a polymer; and
an ion exchange material,
wherein
the MWCNT's form a network of interconnected fibers at the surface of the membrane, which is a capacitive/electrical double-layer (EDL) transducer that stabilizes an electrode potential, and
the solid-contact Al ion-selective electrode is free of an internal filling solution.

2. The solid-contact Al ion selective electrode according to claim 1, wherein the ion exchange material is lipophilic, having a lipophilicity value log P of 10 or greater.

3. The solid-contact Al ion selective electrode according to claim 2, wherein the ion exchange material is potassium tetrakis(4-chlorophenyl)borate (KTCPB).

4. The solid-contact Al ion selective electrode according to claim 1, wherein the MWCNT's comprise 90% carbon or greater and have a length of from 2.5 to 20 μm.

5. The solid-contact Al ion selective electrode according to claim 1, wherein the plasticizer has a dielectric constant of 10 or less.

6. The solid-contact Al ion selective electrode according to claim 5, wherein the plasticizer is nitrophenyl octyl ether (NOPE).

7. The solid-contact Al ion selective electrode according to claim 1, wherein the substrate is selected from the group consisting of glass, a polymer sheet, and a metal foil.

8. The solid-contact Al ion selective electrode according to claim 1, wherein the layer of electrically conductive carbon material comprises one or more selected from the group consisting of carbon black, carbon nanotubes, graphite, and graphene.

9. The solid-contact Al ion selective electrode according to claim 8, wherein the layer of an electrically conductive carbon material further comprises a polymer binder.

10. The solid-contact Al ion selective electrode according to claim 9, wherein the polymer binder is one or more selected from the group consisting of a polytetrafluoroethylene (PTFE), a polyvinyl alcohol (PVA), a polyvinylidene fluoride (PVDF), poly(3,4-ethylenedioxythiophene):poly[(4-styrenesulfonyl) (trifluoromethylsulfonyl)imide] (PEDOT:PSSTFSI), a sodium alginate (SA), a polyacrylic acid (PAA), a sodium carboxymethyl cellulose (CMC) and a styrene-butadiene rubber (SBR).

11. The solid-contact Al ion selective electrode according to claim 1, wherein the polymer contained in the ion selective membrane is selected from the group consisting of polyethylene, polypropylene, and polyvinyl chloride.

12. The solid-contact Al ion selective electrode according to claim 1, wherein the ion selective membrane comprises:
from 0.25 wt % to 5.0 wt % quercetin;
from 2.0 wt % to 10 wt % of the multi-walled carbon nanotubes (MWCNT's);
from 30 wt % to 60 wt % of the plasticizer;
from 20 wt % to 50 wt % of the polymer; and
from 0.05 wt % to 0.50 wt % of the ion exchange material.

13. The solid-contact Al ion selective electrode according to claim 1, wherein the layer of an electrically conductive carbon material comprises from 60 wt % to 80 wt % of the electrically conductive carbon material.

14. A potentiometric aluminum ion sensor, comprising:
a planar substrate;
a screen-printed solid-contact Al ion-selective electrode on the planar substrate;
a screen-printed Ag/AgCl reference electrode electrically isolated from the solid-contact Al ion-selective electrode on the substrate;
wherein
each electrode is configured to be placed in electrical contact with a potentiometer,
the screen printed solid-contact Al ion selective electrode comprises:
a layer of an electrically conductive carbon material applied to a surface of the substrate;
an ion-selective membrane in contact with the layer of electrically conductive carbon material, the ion-selective membrane comprising:
quercetin;
multi-walled carbon nanotubes (MWCNT's);
a plasticizer;
a polymer; and
an ion exchange material,
wherein the MWCNT's form a network of interconnected fibers at the surface of the membrane, which is a capacitive/electrical double-layer (EDL) transducer that stabilizes an electrode potential, and
the solid-contact Al ion-selective electrode is free of an internal filling solution.

15. The potentiometric aluminum ion sensor according to claim 14, wherein the potentiometer is portable to allow for measurement in the field.

16. The potentiometric aluminum ion sensor according to claim 14, wherein the ion exchange material is lipophilic, having a lipophilicity value log P of 10 or greater.

17. The potentiometric aluminum ion sensor according to claim 16, wherein the ion exchange material is potassium tetrakis(4-chlorophenyl)borate (KTCPB).

18. The potentiometric aluminum ion sensor according to claim 14, wherein the MWCNT's comprise 90% carbon or greater and have a length of from 2.5 to 20 μm.

19. The potentiometric aluminum ion sensor according to claim 14, wherein the plasticizer has a dielectric constant of 10 or less.

20. The potentiometric aluminum ion sensor according to claim 19, wherein the plasticizer is nitrophenyl octyl ether (NOPE).

21. The potentiometric aluminum ion sensor according to claim 14, wherein the planar substrate is selected from the group consisting of glass, a polymer sheet, and a metal foil.

22. The potentiometric aluminum ion sensor according to claim 14, wherein the layer of electrically conductive carbon material comprises one or more selected from the group consisting of carbon black, carbon nanotubes, graphite, and graphene.

23. The potentiometric aluminum ion sensor according to claim 22, wherein the layer of an electrically conductive carbon material further comprises a polymer binder.

24. The potentiometric aluminum ion sensor according to claim 23, wherein the polymer binder is one or more selected from the group consisting of a polytetrafluoroethylene (PTFE), a polyvinyl alcohol (PVA), a polyvinylidene fluoride (PVDF), poly(3,4-ethylenedioxythiophene):poly[(4-styrenesulfonyl) (trifluoromethylsulfonyl)imide] (PEDOT:PSSTFSI), a sodium alginate (SA), a polyacrylic acid (PAA), a sodium carboxymethyl cellulose (CMC) and a styrene-butadiene rubber (SBR).

25. The potentiometric aluminum ion sensor according to claim 14, wherein the polymer contained in the ion-selective membrane is selected from the group consisting of polyethylene, polypropylene, and polyvinyl chloride.

26. The potentiometric aluminum ion sensor according to claim 14, wherein the ion-selective membrane comprises:
from 1.0 wt % to 5.0 wt % quercetin;
from 2.0 wt % to 10 wt % of the multi-walled carbon nanotubes (MWCNT's);
from 30 wt % to 60 wt % of the plasticizer;
from 20 wt % to 50 wt % of the polymer; and
from 0.05 wt % to 0.50 wt % of the ion exchange material.

27. The potentiometric aluminum ion sensor according to claim 14, wherein the layer of an electrically conductive carbon material comprises from 60 wt % to 80 wt % of the electrically conductive carbon material.

28. A method for detecting aluminum ion concentration in a sample to be analyzed, comprising:
digesting the sample in a mixture of nitric acid and hydrogen peroxide, followed by microwave digestion;
adjusting the pH of the digested sample to a pH of from 3.0 to 5.0;
electrically connecting a potentiometric aluminum ion sensor according to claim 14 to a potentiometer;
contacting the digested sample with the potentiometric aluminum ion sensor connected to the potentiometer to obtain a potential emf value;
comparing the obtained emf value to a calibration curve of emf value versus log aluminum ion (Al³⁺) in M to determine the Al³⁺ M value of the sample.

29. The method according to claim 28, wherein the pH-adjusted, digested sample is directly contacted with the potentiometric aluminum ion sensor without a purification treatment.

30. The method according to claim 28, wherein the sample to be analyzed is a food or a beverage.

31. The method according to claim 28, wherein a concentration range of Al³⁺ ion is from 0.008 to 1070 μM.

32. The method according to claim 28, wherein a response time of the potentiometric aluminum ion sensor to obtain the Al³⁺ concentration is from 6 to 10 seconds.

33. A method for detecting aluminum ion concentration in a biological fluid to be analyzed, comprising:
adjusting the pH of the biological fluid to 4.5;
electrically connecting a potentiometric aluminum ion sensor according to claim 14 to a potentiometer;
contacting the pH-adjusted sample with the potentiometric aluminum ion sensor connected to the potentiometer to obtain a potential emf value;
comparing the obtained emf value to a calibration curve of emf value versus log aluminum ion (Al³⁺) in M to determine the Al³⁺ M value of the sample.

34. The method according to claim 33, wherein the pH-adjusted sample is directly contacted with the potentiometric aluminum ion sensor without a purification treatment.

35. The method according to claim 33, wherein a response time of the potentiometric aluminum ion sensor to obtain the Al3+ concentration is from 3 to 15 seconds, depending on matrix conditions.
